(19) United States
(12) Patent Application Publication
(10) Pub. No.: US 2025/0049376 A1
(43) Pub. Date: Feb. 13, 2025

(54) Method for Recognizing Motor Imagery Electroencephalography (MI-EEG) Signal Based on Capsule Network (CAPSNET)

(71) Applicant: Dalian University, Liaoning (CN)

(72) Inventors: Xiuli DU, Liaoning (CN); Meiya KONG, Liaoning (CN); Yana LV, Liaoning (CN); Shaoming QIU, Liaoning (CN)

(21) Appl. No.: 18/719,628

(22) PCT Filed: Aug. 16, 2023
(86) PCT No.: PCT/CN2023/113273

§ 371 Date: Jun. 13, 2024

(30) Foreign Application Priority Data
Sep. 5, 2022 (CN) 202211077974.0

(51) Int. Cl. A61B 5/369

(52) U.S. Cl. CPC A61B 5/369

(57) ABSTRACT

A method for recognizing a motor imagery electroencephalography (MI-EEG) signal based on a capsule network (CapsNet) is provided, and relates to the technical field of deep learning and brain-computer interfaces (BCIs). An electroencephalography (EEG) time series is mapped into a three-dimensional (3D) array form based on a spatial electrode distribution. By combining 3D convolution, a CapsNet constructs a three-dimensional capsule network (3D-CapsNet) for recognizing an MI-EEG signal. A 3D convolution module performs feature extraction from both a temporal dimension and an inter-channel spatial dimension through a plurality of layers of 3D convolution to obtain a low-level feature. The low-level feature output by the 3D convolution module is integrated through the CapsNet to obtain a high-level spatial vector containing an inter-feature relationship. A primary capsule and a motor capsule are connected through dynamic routing, and finally a CapsNet module outputs a classification result through a nonlinear activation function squash.

### CROSS REFERENCE TO RELATED APPLICATION

The present application is a national stage application of International Patent Application No. PCT/CN2023/113273, filed on Aug. 16, 2023, which claims priority to the Chinese Patent Application No. 202211077974.0, filed with the China National Intellectual Property Administration (CNIPA) on Sep. 5, 2022, and entitled “METHOD FOR RECOGNIZING MOTOR IMAGERY ELECTROENCEPHALOGRAPHY (MI-EEG) SIGNAL BASED ON CAPSULE NETWORK (CAPSNET)”, which is incorporated herein by reference in its entirety.

### TECHNICAL FIELD

The present disclosure relates to the technical field of deep learning and brain-computer interfaces (BCIs), and specifically, to a method for recognizing a motor imagery electroencephalography (MI-EEG) signal based on a capsule network (CapsNet).

### BACKGROUND

A BCI allows people to interact with the real world solely through a neural activity in a brain. An MI-EEG is one of widely used paradigms of the BCI, and is mainly applied in a field of exercise rehabilitation now. In MI-EEG-based rehabilitation training, on one hand, auxiliary rehabilitation devices such as a wheelchair and a robotic arm are controlled by processing and transforming the MI-EEG in a certain way to convert a motor intention into an instruction. This solves a problem of communication between a patient with a slightly damaged muscle or nerve and an environment to some extent. On the other hand, functional compensation can be achieved by promoting brain function remodeling, thereby ultimately restoring some motor functions, and improving quality of life of the patient.

MI-EEG recognition is a key to improving performance of the BCI. Based on event related synchronization (ERS) and event related desynchronization (ERD), a large number of Ml-EEG classification methods have been proposed successively [1-6]. At present, there are mainly two types of EEG signal recognition technologies: recognition combining traditional manual feature extraction with a machine learning algorithm; and feature extraction and recognition based on a deep learning training model. The method combining the feature extraction and the machine learning has been successfully applied to MI-EEG classification. However, this method divides the feature extraction and classification into the two stages, which makes a parameter of a feature extraction model and a classifier be trained using different objective functions. In addition, the obtaining of the optimal feature is subjective. If a suboptimal feature combination is selected during the feature extraction, the classification performance will be affected. Above all, for a complex nonlinear random time series, a method for manually determining a feature greatly relies on expert experience and an understanding of an EEG by an expert. Due to the differences among the different subjects, a method for selecting a feature for each subject cannot be well applied to the larger population.

Recently, a plurality of deep learning methods have been applied to EEG classification, such as a convolutional neural network (CNN) [7], a recurrent neural network (RNN) [8], and a CapsNet [9]. When the EEG signal is recognized directly through deep learning, it is not required to manually extract the feature contained in the EEG signal, and the feature extraction and classification are embedded into the end-to-end network, achieving joint parameter optimization. This minimizes the preprocessing process of the EEG signal and is obviously more suitable for the online BCI research. During the MI-EEG classification through the deep learning, a primary task is to represent the MI-EEG in a form that can be processed by a deep model. In addition, the research on the EEG signal recognition needs to overcome the problems of the small dataset and the unclear EEG feature, and has a strict requirement for a recognition method because it is necessary to fully extract the contained feature while avoiding the overfitting.

At present, the MI-EEG is usually represented in a form of a two-dimensional (2D) matrix, which is hereinafter referred to as 2DMI-EEG. This representation method uses a quantity of sampled electrodes as a height and a sampling time step as a width. Another common method is to transform the EEG signal into a 2D time-frequency image as a network input through short-time Fourier transform, wavelet transform, or the like. However, the representation method using the 2D matrix or the 2D time-frequency image cannot retain spatial information of the MI-EEG, and an inherent relationship between adjacent electrodes cannot be reflected in the 2D matrix. This will affect the classification performance. In 2015, Bashivan et al. [10] proposed a method for retaining original spatial, spectral, and temporal structures of an EEG. In this method, a power spectrum of an EEG signal of each electrode is firstly calculated. Then a sum of squares of absolute values of three selected frequency bands is obtained. Finally, an electrode distribution diagram is mapped as an input image of a model by using an azimuthal equidistant projection (AEP) method. Based on such representation method, recognition performance is significantly improved. This indicates that a spatial feature is extremely important for an EEG-based classification task.

However, most of the above two major types of research methods only focus on recognition of an EEG signal represented in a two-dimensional (2D) form, and fail to fully reflect spatial information contained in the EEG signal. Collected from a three-dimensional (3D) scalp surface, an MI-EEG signal is a nonlinear random time series with spatio-temporal information. Therefore, when the EEG signal is processed, it is more reasonable to consider its temporal and spatial characteristics.

### Existing Technical Solutions

In 2019, Zhao et al. [11]proposed a 3D representation method for the EEG signal to map an EEG time series into a 3D array as a model input based on a spatial electrode distribution. This method can retain both a temporal feature and the spatial feature. In addition, a multi-branch three-dimensional convolutional neural network (3DCNN) was proposed to classify a 3DMI-EEG. The 3DCNN extracts an MI-related feature by using three branches with different receptive fields. The three branches are referred to as a small receptive field (SRF) network, a medium receptive field (MRF) network, and a large receptive field (LRF) network. Finally, a fully connected layer is combined with Softnax for classification. This is a successful attempt in classifying raw EEG data. Afterwards, Liu et al. [12] conducted further research on this basis. A three-branch structure is still used, and a dense connection mode is introduced to improve 3DMI-EEG classification of the multi-branch 3DCNN. This overcomes the overfitting to a certain extent while deepening the network, thereby improving performance to some extent.

For an EEG signal represented in a 3D form, a CNN is used in both references [11] and [12]. In order to retain more features of the EEG signal, no pooling layer is used for dimensionality reduction, and a multi-branch structure is used, resulting in a relatively large quantity of network parameters. In addition, although both the temporal feature and the spatial feature are considered, an inherent relationship between features cannot be expressed by the network, which affects the recognition performance to some extent.

### CITED REFERENCES

[1] BOSTANOV V.BCI competition 2003-data sets Ib and IIb:feature extraction from event-related brain potentials with the continuous wavelettransform and the t-value scalo-gram[J]. IEEE Transactions on Biomedicalengineering, 2004, 51(6):1057-1061.
[2] HSU W Y, SUN Y N. EEG-based motor imagery analysis using weighted wavelet transform features[J]. Journal of neuroscience methods, 2009, 176(2):310-318.
[3] BURKE D P, KELLY S P, D E CHAZAL P, et al. A parametric feature extraction and classification strategy for brain-computer interfacing [J]. IEEE Transactions on Neural Systems and Rehabilitation Engineering, 2005, 13(1):12-17.
[4] RAMOSER H, MULLER-GERKING J, PFURTSCHELLER G. Optimal spatial filtering of single trial EEG during im-agined hand movement [J]. IEEE transactions on rehabilitation engineering, 2000, 8(4):441-446.
[5] ANG K K, CHIN Z Y, WANG C, et al. Filter bank common spatial pattern algorithm on BCI competition IV datasets 2a and 2b [J]. Frontiers in neuroscience, 2012, 6:39.
[6] NOVI Q, GUAN C, DAT T H, et al. Sub-band common spatial pattern (SBCSP) for brain-computer inter-face [C]/12007 3rd International IEEE/EMBS Conference on Neural Engineering. IEEE, 2007:204-207.
[7] LI M A, HAN J F, DUAN L J. A novel MI-EEG imaging with the location information of electrodes [J]. IEEE Access, 2019, 8:3197-3211.
[8] ABBASVANDI Z, NASRABADI A M. A self-organized recurrent neural network for estimating the effective con-nectivity and its application to EEG data [J]. Computers in biology and medicine, 2019, 110:93-107.
[9] Chen Qin, Chen Lanlan, Jiang Runqiang. EEG Emotion Recognition for Integrated CapsNet [J]. Computer Engineering and Applications 2022, 58(08):175-184.CHEN QIN, CHEN LANLAN, JIANG RUNQIANG. Emo-tion recognition of EEG based on Ensemble CapsNet[J]. CEA, 2022, 58(08):175-184.
[10] BASHIVAN P, RISH I, YEASIN M, et al. Learning repre-sentations from EEG with deep recurrent-convolutional neural networks [J]. arXiv preprintar Xiv:1511.06448, 2015.
[11] ZHAO X, ZHANG H, ZHU G, et al. A multi-branch 3D convolutional neural network for EEG-based motor im-agery classification [J]. IEEE transactions on neural systems and rehabilitation engineering, 2019, 27(10):2164-2177.
[12] LIU T, YANG D. A Densely Connected Multi-Branch 3D Convolutional Neural Network for Motor Imagery EEG Decoding [J]. Brain Sciences, 2021, 11(2):197.

### SUMMARY

In order to solve the above problems, the present disclosure provides a method for recognizing an MI-EEG signal based on a CapsNet, including the following steps:

S1: mapping an EEG time series of an MI-EEG signal into a 3D array form based on a spatial electrode distribution;

S2: constructing, by using a CapsNet and 3D convolution, a three-dimensional capsule network (3D-CapsNet) model for recognizing the MI-EEG signal, and using an EEG signal in the 3D array form described in the S1 as an input of the 3D-CapsNet model for recognizing the MI-EEG signal, where a 3D-CapsNet includes a 3D convolutional module and a CapsNet module; the 3D convolution module performs feature extraction on the input EEG signal in the 3D array form from both a temporal dimension and an inter-channel spatial dimension through a plurality of layers of 3D convolution to obtain a low-level feature; and the CapsNet module has a spatial detection capability, and the low-level feature output by the 3D convolution module is integrated through the CapsNet to obtain a high-level spatial vector containing an inter-feature relationship; and

S3: training the CapsNet module by using a dynamic routing algorithm, connecting a primary capsule and a motor capsule through dynamic routing, and finally outputting a classification result through a nonlinear activation function squash.

The present disclosure has the following beneficial effects: The present disclosure combines the 3D convolution to propose the 3D-CapsNet model for recognizing the MI-EEG signal, which overcomes an individual difference to a certain extent while improving recognition accuracy. The 3D-CapsNet comprehensively considers a temporal dimension and a channel spatial dimension of an MI-EEG and an inter-feature intrinsic relationship to maximize a feature expression capability of the network. In addition, the inter-capsule dynamic routing-based connection method of the CapsNet replaces a traditional fully connected layer. In this way, the network does not need to use a pooling layer to reduce feature dimensions. Therefore, many detailed EEG features are retained, ensuring effective feature extraction.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other accompanying drawings can be derived from these accompanying drawings by those of ordinary skill in the art without creative efforts.

FIG. 1 is a schematic diagram of a 3D representation process of an MI-EEG according to an embodiment of the present disclosure, where (a) of FIG. 1 shows an electrode montage of an international 10-20 system; (b) of FIG. 1 shows TP 2D matrices; and (c) of FIG. 1 shows an MI-EEG signal represented in a 3D form;

FIG. 2 is a structural diagram of a 3D-CapsNet according to an embodiment of the present disclosure;

FIG. 3A-3C are schematic diagrams of different network structures involved in an experimental process according to an embodiment of the present disclosure, where FIG. 3A shows a 3D-CapsNet; FIG. 3B shows a 3DCNN; and FIG. 3C shows a 2D-CapsNet;

FIG. 4 illustrates an inter-capsule information transfer and routing process according to an embodiment of the present disclosure; and

FIG. 5A-5C are schematic diagrams of a confusion matrix according to an embodiment of the present disclosure.

### DETAILED DESCRIPTION OF THE EMBODIMENTS

A particular embodiment of the present disclosure will be described below, and other advantages and effects of the present disclosure will become apparent for those skilled in the art from the disclosure of this specification. In order to better understand the objective, structure and function of the present disclosure, a method for recognizing an MI-EEG signal based on a CapsNet in the present disclosure is described in further detail below with reference to the accompanying drawings.

Inspired by a dynamic routing-based connection method of a CapsNet, a 3D-CapsNet model for recognizing the MI-EEG signal is proposed by combining 3D convolution. A 3D convolution module performs feature extraction from both a temporal dimension and an inter-channel spatial dimension through a plurality of layers of 3D convolution to obtain a low-level feature. The CapsNet also has a certain spatial detection capability. The low-level feature output by the 3D convolution module is integrated through the CapsNet to obtain a high-level spatial vector containing an inter-feature relationship. Finally, a classification result is output through a nonlinear activation function squash. The 3D-CapsNet comprehensively considers a temporal feature and a spatial feature of an original EEG signal, adopts the dynamic routing-based connection method, abandons a pooling layer to retain a subtle feature, and maximizes a feature expression capability of the network. A method for recognizing an MI-EEG signal based on a CapsNet is provided. A specific implementation is as follows:

### Embodiment 1

A method for recognizing an MI-EEG signal based on a CapsNet includes the following steps:

S1: Map an EEG time series into a 3D array form based on a spatial electrode distribution.

S2: Construct, by using a CapsNet and 3D convolution, a 3D-CapsNet model for recognizing an MI-EEG signal, and use an EEG signal in the 3D array form described in the S1 as an input of the recognition model. The 3D-CapsNet includes a 3D convolution module and a CapsNet module. The 3D convolution module performs feature extraction on the input EEG signal in the 3D array form from both a temporal dimension and an inter-channel spatial dimension through a plurality of layers of 3D convolution to obtain a low-level feature. The CapsNet module has a spatial detection capability, and the low-level feature output by the 3D convolution module is integrated through the CapsNet to obtain a high-level spatial vector containing an inter-feature relationship.

S3: Train the CapsNet module by using a dynamic routing algorithm, connect a primary capsule and a motor capsule through dynamic routing, and finally output a classification result through a nonlinear activation function squash.

The step S1 includes the following substeps:

Intercepting the EEG signal by frame, obtaining a value of a current frame, transforming a value of each frame into an x×y 2D-map based on a general spatial distribution of a sampled electrode, and filling an unused electrode position with 0.

Expanding TP 2D-maps into an x×y×TP 3D matrix based on time series information of the EEG signal, where TP represents a quantity of sampling points for each channel, and TP is a natural number.

The step S2 is executed as follows: constituting the 3D convolution module by encapsulating five 3D convolution layers, such that the 3D convolution module can extract a basic feature of data at a plurality of levels to provide local perceptual information for a main capsule layer, and gradually increasing a quantity of convolution kernels to ensure that increasingly rich features are correctly extracted; performing batch normalization (BN) after each convolution to accelerate convergence and reduce overfitting; inputting the input into the convolution module to generate 128 4*5*6 outputs, converting the outputs into a 128*4*5*6 tensor, and sending the 128*4*5*6 tensor to the main capsule layer, such that the main capsule layer outputs 384 4-dimensional capsules, where the main capsule stores spatial features of different forms for an MI-EEG signal; connecting the main capsule layer and a motor capsule layer through the dynamic routing; aggregating, by the dynamic routing algorithm, predicted capsules that are similar to each other, and obtaining, through abstraction, a motor capsule capable of representing an inter-class difference; and outputting the classification result through the nonlinear activation function squash.

The step S3 is specifically as follows: training the CapsNet by using the dynamic routing algorithm, where an inter-capsule information transfer and routing process is only carried out between two consecutive capsule layers, that is, the dynamic routing algorithm is used between ûᵢⱼ and sⱼ. A specific process is as follows:

$${\hat{u}}_{ij} = {u}_{i} {W}_{ij} \qquad ( 1 )$$

firstly, defining uᵢ(i=1, 2, . . . , n) to represent a detected low-level feature vector, and multiplying the low-level feature vector uᵢ by a corresponding weight matrix Wᵢⱼ to obtain a high-level output vector ûᵢⱼ, where i represents an iₜₕ low-level feature, and j represents a jₜₕ primary capsule; as shown in the formula (1), encoding a probability of a corresponding feature based on a vector length, and encoding an internal status of the feature based on a vector direction; and performing the above steps to encode a spatial relationship between the low-level feature and a high-level feature, where ûᵢⱼ is also referred to as the primary capsule;
secondly, weighting the primary capsule ûᵢⱼ such that the capsule learns a coupled sparse weight cᵢⱼ by using the dynamic routing algorithm; adjusting the cᵢⱼ, and sending, by the primary capsule ûᵢⱼ, an output to an appropriate motor capsule sⱼ, where the sⱼ is a result of performing weighted summation on predicted vectors of a plurality of primary capsules, predicted values that are similar to each other are aggregated, and an entire process is shown in a formula (2):

$${s}_{j} = {Σ}_{i} {\hat{u}}_{ij} {c}_{ij} ; \qquad ( 2 )$$

and
finally, processing the sⱼ by using the nonlinear activation function squash, such that a length is compressed to within 0 to 1 without changing the vector direction, and a result is represented as a vector vⱼ, where as shown in a formula (3), the probability of the corresponding feature is encoded based on the vector length, and the internal status of the feature is encoded based on the vector direction:

$${v}_{j} = \frac{‖ {s}_{j} {‖}^{2}}{1 + ‖ {s}_{j} ‖} \cdot \frac{{s}_{j}}{‖ {s}_{j} ‖} \qquad ( 3 )$$

The above three steps are a complete inter-capsule propagation process, where learning of a coupling coefficient Cᵢⱼ is an essence of the dynamic routing algorithm, and the coupling coefficient is determined according to the following formula (4):

$${c}_{ij} = \frac{exp ( {b}_{ij} )}{{Σ}_{k} exp ( {b}_{ik} )} . \qquad ( 4 )$$

In the above formula, bᵢⱼ represents a temporary variable with an initial value of 0. After a first iteration, all values of the coupling coefficient Cᵢⱼ are equal. As the iteration progresses, a value of the bᵢⱼ is updated, and a uniform distribution of the cᵢⱼ changes. The bᵢⱼ is updated according to the following formula (5):

$${b}_{ij} ← {b}_{ij} + {\hat{u}}_{ij} \cdot {v}_{j} \qquad ( 5 )$$

3D representation of an MI-EEG:

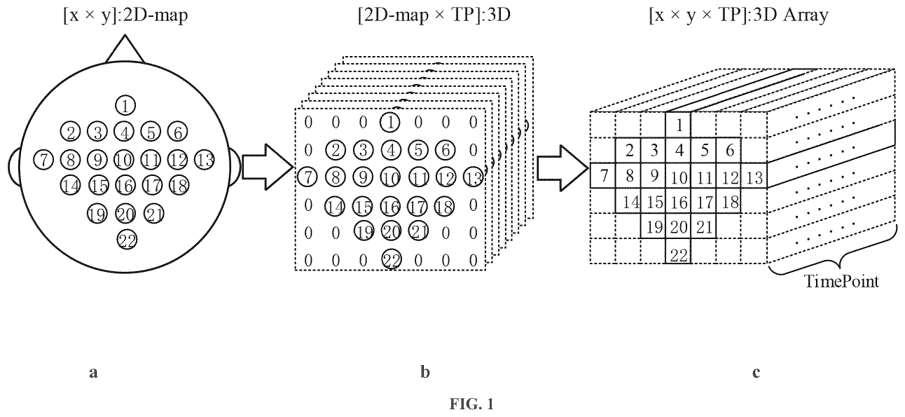

FIG. 1 shows a mapping process of a tested 3D MI-EEG signal in BCI competition IV datasets 2a. At first, the EEG signal is intercepted by frame, the value of the current frame is obtained, the value of each frame is transformed into the 2D matrix (2D-map) based on the general spatial distribution of the sampled electrode, and the unused electrode position is filled with 0. Then, the TP 2D-maps are expanded to the x×y×TP 3D matrix based on the temporal information of the EEG signal, where TP represents the quantity of sampling points of each channel. This method for representing the MI-EEG in the 3D form based on an electrode distribution not only fully retains temporal information present in the EEG time series, but also retains spatial information present in the electrode distribution, while ensuring processability of EEG data.

### Hierarchical Structure of the 3D-CapsNet:

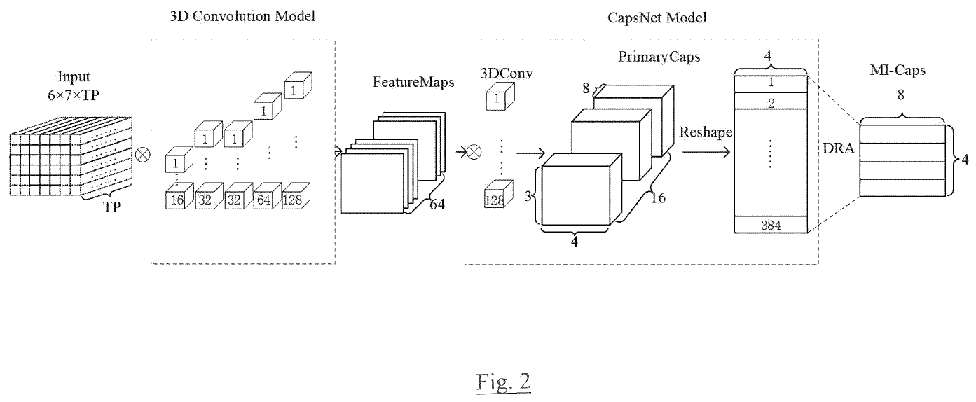

The 3D-CapsNet is mainly constituted by the 3D convolution module and the CapsNet module. A framework of the 3D-CapsNet is shown in FIG. 2. Firstly, the feature extraction is performed from the temporal dimension and the inter-channel spatial dimension through the multi-layer 3D convolution, and the primary feature is obtained through the abstraction. Secondly, an inter-feature intrinsic relationship is detected by using a convolutional capsule layer. Finally, the high-dimensional feature vector is obtained through dynamic routing-based connection, which is known as the motor capsule, and then the motor capsule is classified in combination with the function squash.

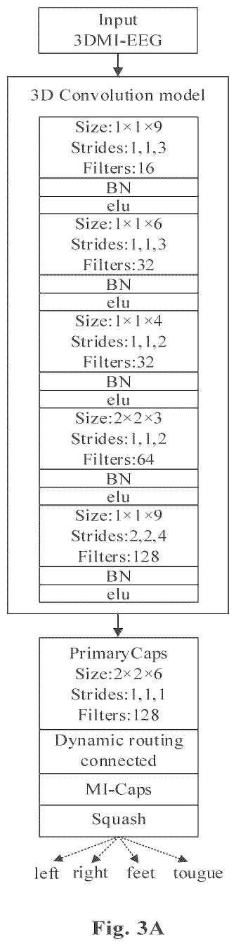

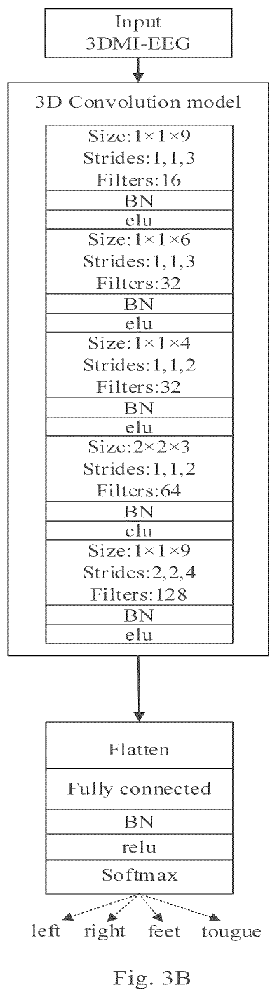

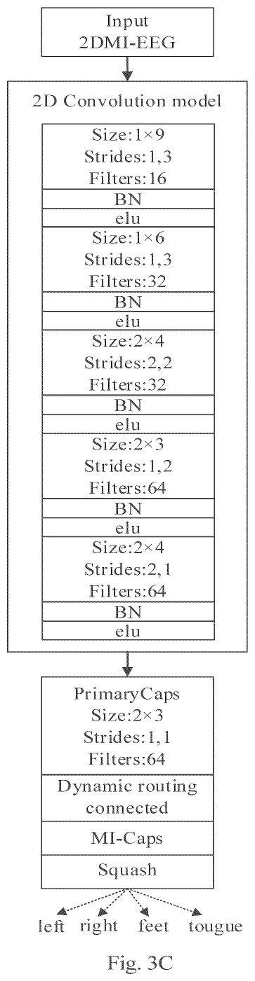

Specific parameters of the hierarchical structure of the 3D-CapsNet are shown in FIG. 3A, and the parameters are set to optimal values obtained through continuous tentative experimentation. The 3D convolution module can extract the basic feature of the data at the plurality of levels to provide the local perceptual information for the main capsule layer. The quantity of convolution kernels gradually increases to ensure that the increasingly rich features are correctly extracted. The BN is performed after each convolution to accelerate the convergence and reduce the overfitting. The input is input into the convolution module to generate the 128 4*5*6 outputs, the outputs are converted into the 128*4*5*6 tensor, and the 128*4*5*6 tensor is sent to the main capsule layer. The main capsule layer outputs the 384 4-dimensional capsules, and the main capsule stores the spatial features of different forms for the MI-EEG signal. The main capsule layer and the motor capsule layer are connected through the dynamic routing. The dynamic routing algorithm aggregates the predicted capsules that are similar to each other, and the motor capsule capable of representing the inter-class difference is obtained through the abstraction. Finally, the classification result is output through the nonlinear activation function squash.

### Experimental Environment:

The 3D CapsNet model is implemented by using Python under a Python framework. The experimental environment is as follows: 11th Gen Intel (R) Core (TM) i5-11400H@2.70 GHz 2.69 GHz, 16 GB memory, NVIDIA GeForceRTX3050 graphics card, and 64-bit Windows 11 system.

### Training Algorithm and Training Strategy:

Training algorithm: The CapsNet is trained by using the dynamic routing algorithm. FIG. 4 shows the inter-capsule information transfer and routing process. The dynamic routing algorithm is only used between the two consecutive capsule layers (ûᵢⱼ and sⱼ). Firstly, uᵢ(i=1, 2, . . . , n) represents the detected low-level feature vector. The low-level feature vector uᵢ is multiplied by the corresponding weight matrix Wᵢⱼ to obtain the high-level output vector ûᵢⱼ. As shown in the following formula (1), the probability of the corresponding feature is encoded based on the vector length, and the internal status of the feature is encoded based on the vector direction. The ûᵢⱼ is also referred to as the primary capsule. This step encodes spatial and other important relationships between the low-level feature and the high-level feature.

$${\hat{u}}_{ij} = {u}_{i} {W}_{ij} \qquad ( 1 )$$

In the above formula, i represents the iₜₕ low-level feature, and j represents the jₜₕ primary capsule.

Secondly, the primary capsule ûᵢⱼ is weighted. This step is similar to scalar weighting in a neuron, except that a weight of the neuron is learned by using a backpropagation algorithm, while the capsule uses the dynamic routing algorithm to learn the coupled sparse weight Cᵢⱼ The cᵢⱼ is adjusted, and the primary capsule sends the output to the appropriate motor capsule sⱼ. The sⱼ is the result of performing the weighted summation on the predicted vectors of the primary capsules, and the predicted values that are similar to each other are aggregated. The entire process is shown in the following formula (2):

$${s}_{j} = {\sum }_{i} {\hat{u}}_{ij} {c}_{ij} \qquad ( 2 )$$

Finally, the sⱼ is processed by using the nonlinear activation function squash, such that the length is compressed to within 0 to 1 without changing the vector direction, and the result is represented as the vector vⱼ. As shown in the following formula (3), the probability of the corresponding feature is encoded based on the vector length, and the internal status of the feature is encoded based on the vector direction.

$${v}_{j} = \frac{{‖ {s}_{j} ‖}^{2}}{1 + ‖ {s}_{j} ‖} \cdot \frac{{s}_{j}}{‖ {s}_{j} ‖} \qquad ( 3 )$$

The above three steps are the complete inter-capsule propagation process between. The learning of the coupling coefficient cᵢⱼ is the essence of the dynamic routing algorithm, and the coupling coefficient is determined according to the following formula (4):

$${c}_{ij} = \frac{exp ( {b}_{ij} )}{{\sum }_{k} exp ( {b}_{ik} )} . \qquad ( 4 )$$

In the above formula, bᵢⱼ represents the temporary variable with the initial value of 0. After the first iteration, all the values of the coupling coefficient cᵢⱼ are equal. As the iteration progresses, the value of the bᵢⱼ is updated, and the uniform distribution of the cᵢⱼ changes. The bᵢⱼ is updated according to the following formula (5):

$${b}_{ij} ← {b}_{ij} + {\hat{u}}_{ij} \cdot {v}_{j} \qquad ( 5 )$$

A capsule loss is evaluated by using a margin loss function, which is represented as Lₖ. For each category k, the Lₖ is calculated according to the following formula:

$${L}_{k} = {T}_{k} max {( 0 , {m}^{+} - ‖ {v}_{k} ‖ )}^{2} + λ ( 1 - {T}_{k} ) max {( 0 , ‖ {v}_{k} ‖ - {m}^{-} )}^{2} \qquad ( 6 )$$

When and only when there is a motor imagery of the category k, Tₖ=1, m⁺=0.9, and m⁻=0.1. A value of λ is set to an empirical value 0.5 to reduce losses of some categories that do not appear. A total loss is a sum of losses of all motor capsules.

Training strategy: The present disclosure adopts a cropped training strategy. During cropped training, a sample is generated by sliding a 3D window along the temporal dimension with a certain data step. The window covers all electrodes. A size of the window in the temporal dimension is related to an EEG data sampling frequency and a specific task. The cropped training strategy for the EEG signal is a common method for enhancing a training sample of the EEG signal, which is similar to a cropping strategy in a field of image recognition. A plurality of experiments have shown that cropped sample training has better classification performance compared with complete sample training. The CapsNet is trained by optimizing the margin loss function, with a quantity of training iterations set to 80. A learning rate is dynamically adjusted by using an Adam random optimization algorithm, which can replace a classic stochastic gradient descent (SGD) process to update a network weight more effectively and accelerate convergence of a neural network.

### Verification Process:

An experimental verification stage progresses layer by layer. Firstly, effectiveness of applying the CapsNet in EEG signal recognition is verified on the 3D-CapsNet. As shown in FIG. 3A, the 3D-CapsNet monitors test data from nine subjects for 80 training cycles. The 3D-CapsNet performs excellently on data from subjects 1, 3, 7, 8, and 9, achieving high accuracy and overall stability after 40 iterations. The 3D-CapsNet also performs well on subjects 4 and 6. For subjects 2 and 5, although performance of the model is not as ideal as that of the model on data from other subjects, an accuracy of about 70% can still be achieved. Based on overall performance of the 3D-CapsNet on the data of all the subjects, the model does not exhibit a significant bias due to a change in the subjects, and has a certain degree of robustness.

Secondly, excellent performance of the CapsNet is further verified on a 3DCNN structure shown in FIG. 3B. In the experiment, a quantity of iterations when a loss value tends to be stable is used as a quantity of iterations in a subsequent experiment. The quantities of iterations both are set to 80, and the Python is used for implementation. Good performance is achieved under the dynamic routing-based connection for all other subjects except the subject 5. In addition, a lower standard deviation is achieved under the dynamic routing-based connection. Therefore, it can be concluded that the CapsNet is superior to a traditional CNN in MI-EEG recognition.

Finally, performance of an improved network based on the CapsNet on an 2D MI-EEG and the 3D MI-EEG is compared. The 3D convolution in the 3D-CapsNet is replaced with 2D convolution. A framework structure of a 2D-CapsNet is shown in FIG. 3C. A recognition experiment is conducted on an input of a 2DMI-EEG and compared with the method in the present disclosure. For the nine subjects, recognition accuracy achieved when a 3D MI-EEG representation is used is higher than that achieved when a 2D MI-EEG representation is used. From a perspective of a standard deviation, the standard deviation achieved under the 3DMI-EEG representation is lower than that achieved under the 2DMI-EEG representation. In other words, the MI-EEG signal represented in the 3D form is more suitable for decoding using a deep network.

The 3D-CapsNet comprehensively considers the temporal dimension and a channel spatial dimension of the MI-EEG and the inter-feature intrinsic relationship to maximize a feature expression capability of the network. In addition, the inter-capsule dynamic routing-based connection method of the CapsNet replaces a traditional fully connected layer. In this way, the network does not need to use a pooling layer to reduce feature dimensions. Therefore, many detailed EEG features are retained, ensuring effective feature extraction.

### Experimental Result:

Experiments are conducted to compare the method in the present disclosure with similar studies. Among them, a DeepNet, an EEGNet, and a ShallowNet all decode the EEG signal based on the 2D-EEG, while in references [11] and [12], the EEG signal is decoded based on the 3D MI-EEG. Classification accuracy results of different methods on the evaluation datasets of the nine subjects are shown in Table 1. It can be seen that methods in the reference [12] and the present disclosure have certain advantages in recognition accuracy, while a method in the reference [11] has lower decoding accuracy than the EEGNet and the ShallowNet, but has a much smaller standard deviation of accuracy than the EEGNet and the ShallowNet. In general, a standard deviation based on the 3D representation form is much smaller than that based on the 2D representation form. It can be inferred that the 3D representation form of the MI-EEG signal is more suitable for decoding the EEG signal and can improve the recognition accuracy to a certain extent. Such a representation form is more conducive to retaining a common IM-EEG feature of different subjects, which can overcome an individual difference to a certain extent, and achieve stronger interpretability. In addition, the method proposed in this specification generally has higher recognition accuracy than methods described in cutting-edge references, with highest accuracy for datasets of six subjects compared with similar research methods and an average accuracy of 2.805% higher than a suboptimal result.

TABLE 1
Comparison of classification accuracy achieved
in the present disclosure and similar studies
Accuracy/%

| | | | | Reference | Reference | Present |
|---|---|---|---|---|---|---|
| Subject | DeepNet | EEGNet | ShallowNet | [15] | [20] | disclosure |
| 1 | 73.13 | 80.98 | 79.29 | 77.397 | 82.894 | 90.640 |
| 2 | 55.98 | 54.96 | 65.14 | 60.140 | 67.336 | 69.762 |
| 3 | 79.46 | 88.80 | 92.19 | 82.927 | 88.117 | 93.923 |
| 4 | 74.42 | 69.02 | 65.94 | 72.288 | 79.444 | 79.425 |
| 5 | 74.60 | 79.15 | 77.81 | 75.836 | 82.1117 | 74.587 |
| 6 | 54.73 | 72.59 | 71.52 | 68.988 | 74.285 | 76.986 |
| 7 | 67.28 | 84.96 | 93.98 | 76.036 | 86.563 | 88.881 |
| 8 | 83.21 | 86.16 | 86.61 | 76.855 | 81.918 | 89.075 |
| 9 | 83.84 | 91.65 | 89.02 | 84.665 | 88.333 | 92.969 |
| Average value | 71.85 | 78.70 | 80.17 | 75.015 | 81.223 | 84.028 |
| Standard deviation | 10.68 | 11.54 | 10.98 | 7.34 | 6.85 | 8.90 |

To further verify performance of the 3D-CapsNet, a Kappa value in the classification result is calculated and compared with those achieved in the references [11] and [12]. A result is shown in Table 2. The Kappa value is mainly used for consistency testing to measure consistency between a predicted result of the model and an actual classification result. The Kappa value ranges from −1.0 to 1.0, and a larger value indicates better classification performance of an algorithm. An expression of the Kappa value is as follows:

$$Kappa = \frac{{P}_{o} - {P}_{e}}{1 - {P}_{e}} \qquad ( 7 )$$

In the above expression, P_{c} represents total sample classification accuracy, and Pₑ is used to evaluate an accidental probability. Assuming that C represents a total quantity of categories, Tᵢ(i=1, 2, . . . , c) represents a quantity of correctly classified samples for each category, an actual quantity of samples for each category is a₁, a₂, . . . a_{c}, a predicted quantity of samples for each category is b₁, b₂, . . . b_{c}, and a total quantity of samples is n, the following formulas are met:

$${P}_{o} = \frac{{\sum }_{i = 1}^{c} {T}_{i}}{n} \qquad ( 8 )$$

$${P}_{e} = \frac{{\sum }_{i = 1}^{c} {a}_{i} * {b}_{i}}{{n}^{2}} \qquad ( 9 )$$

The result is shown in Table 2. For all other subjects except the subject 5, the Kappa value achieved in the method proposed in this specification is better than Kappa values achieved in the comparative references. Therefore, the 3D-CapsNet has good performance for 3DMI-EEG recognition.

TABLE 2
Comparison of Kappa values achieved in the
present disclosure and similar studies

| | Kappa value | | |
|---|---|---|---|
| Subject | Reference [15] | Reference [20] | Present disclosure |
| 1 | 0.699 | 0.775 | 0.878 |
| 2 | 0.459 | 0.543 | 0.598 |
| 3 | 0.788 | 0.824 | 0.924 |
| 4 | 0.594 | 0.604 | 0.726 |
| 5 | 0.647 | 0.777 | 0.662 |
| 6 | 0.538 | 0.591 | 0.693 |
| 7 | 0.653 | 0.837 | 0.860 |
| 8 | 0.702 | 0.672 | 0.847 |
| 9 | 0.713 | 0.870 | 0.911 |
| Average value | 0.644 | 0.721 | 0.789 |

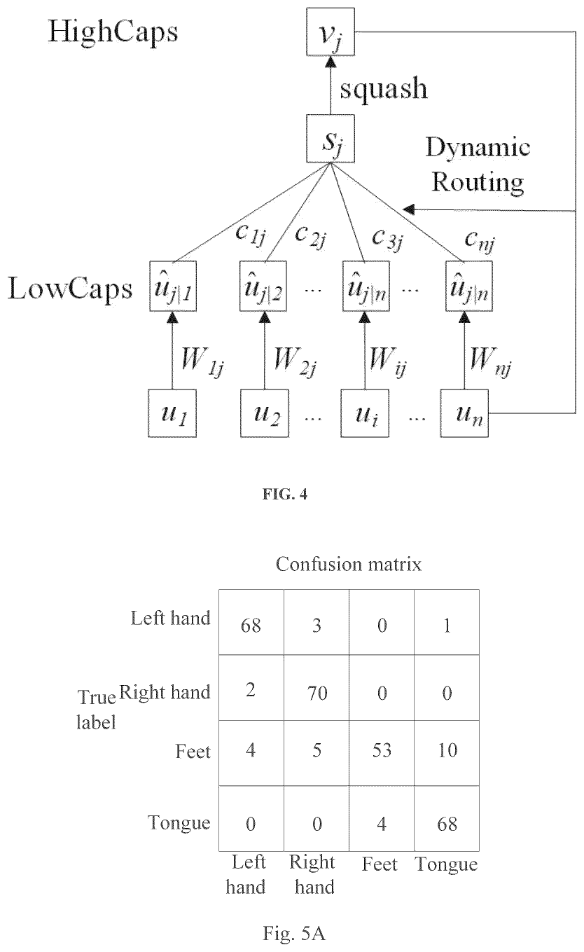

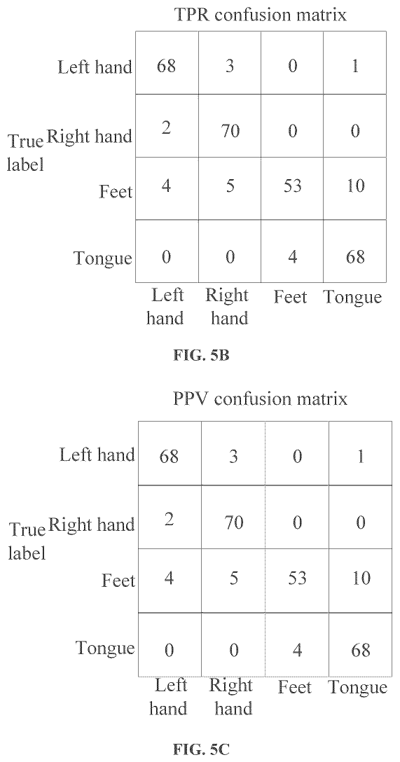

As shown in FIG. 5A-5C, a confusion matrix of the 3D-CapsNet is evaluated. The confusion matrix visualizes a quantity of correct predictions for each category and a quantity of incorrect predictions for other categories. TPR represents sensitivity, which is a proportion of correctly predicted samples to truly labeled samples. PPV represents accuracy, which is a proportion of a total quantity of correctly predicted samples to a total quantity of samples predicted by the model. A comprehensive indicator F1-Score is used for evaluation. The F1-Score is calculated according to the following formula (10). The indicator F1-Score combines output sensitivity and accuracy, with a value ranging from 0 to 1, where 1 represents a best output of the model and 0 represents a worst output of the model. An experimental result shows that values that are of the F1-Score and correspond to a left hand, a right hand, feet, and a tongue are F1—Score_{(left)}=0.93, F1-Score_{(right)}=0.93, F1-Score_{(feet)}=0.82, and F1-Score_{(tongue)}=0.90 respectively. It is obvious that the model has best classification performance for motor imagery of the left and right hands among the four categories, followed by classification performance for motor imagery of the tongue. Classification performance for motor imagery of the feet is slightly inferior, but overall performance of the model is very good.

$$F 1 - Score = \frac{2 TPR \times PPV}{TPR + PPV} \qquad ( 10 )$$

In addition, for an application of a BCI system, it is important to establish a stable classification model that can overcome the individual difference. Once such a model is obtained, any subject can directly use the model for an MI-related classification task without any pre-training. This embodiment of the present disclosure uses mixed “training data” of the nine subjects as a model training set to train the model, and uses corresponding “test data” of each subject as a model test set. The classification performance is shown in Table 3. From the table, it can be seen that the framework proposed in this specification can still achieve effective classification with an average accuracy of 66.922. This means that the proposed framework has a capability of overcoming the individual difference in Ml classification, with average accuracy improved by 17 percentage points compared with that of Multi-Branch3D. Therefore, the method for recognizing an Ml-EEG signal based on a CapsNet effectively improves the capability of overcoming the individual difference.

TABLE 3
Comparison of capabilities of overcoming the
individual difference in the similar studies

Accuracy/%

| Subject | Multi- Branch3D | 3D-CapsNet |
|---|---|---|
| 1 | 49.51 | 68.34 |
| 2 | 40.74 | 57.25 |
| 3 | 64.51 | 80.02 |
| 4 | 44.57 | 61.37 |
| 5 | 54.29 | 62.55 |
| 6 | 40.74 | 65.72 |
| 7 | 58.88 | 74.39 |
| 8 | 59.76 | 75.11 |
| 9 | 56.84 | 78.21 |
| Average value | 52.17 | 69.22 |

The classification accuracy and a time consumption are two major factors that need to be considered in a practical application of the BCI system. Therefore, it is not scientific to solely consider the recognition accuracy. An experimental result of He et al. indicates that a BCI system with a response time of 1 second or less is very suitable for a real-time application. In this embodiment of the present disclosure, an experiment is conducted on a GeForceRTX3050 GPU with a 4 GB graphics memory. Time spent on recognizing the nine subjects is obtained, as shown in Table 4. The result shows that the method for recognizing an MI-EEG signal based on a CapsNet has a fast prediction speed (5.1×10⁻³ s, approximately 1.48 s for 288 samples) and high prediction accuracy, proving that the 3D-CapsNet is more competitive in implementing an online application of the BCI system.

TABLE 4
Test time

| Subject | Time (s) | Accuracy (%) |
|---|---|---|
| 1 | 1.6 | 90.64 |
| 2 | 1.5 | 69.76 |
| 3 | 1.3 | 93.92 |
| 4 | 1.8 | 79.43 |
| 5 | 1.4 | 74.59 |
| 6 | 1.3 | 76.99 |
| 7 | 1.5 | 88.88 |
| 8 | 1.3 | 89.08 |
| 9 | 1.6 | 92.97 |
| Mean | 1.48 | 84.03 |

The above are merely preferred specific implementations of the present disclosure, but the protection scope of the present disclosure is not limited thereto. Any equivalent replacement or modification made by a person skilled in the art according to the technical solutions of the present disclosure and inventive concepts thereof within the technical scope of the present disclosure shall fall within the protection scope of the present disclosure.

Although the present disclosure has been described in detail above with general descriptions and specific embodiments, some modifications or improvements can be made on the basis of the present disclosure, which is apparent to those skilled in the art. Therefore, all of these modifications or improvements made without departing from the spirit of the present disclosure fall within the claimed scope of the present disclosure.

What is claimed is:

1. A method for recognizing a motor imagery electroencephalography (MI-EEG) signal based on a capsule network (CapsNet), comprising the following steps:
S1: mapping an electroencephalography (EEG) time series of an MI-EEG signal into a three-dimensional (3D) array form based on a spatial electrode distribution;
S2: constructing, by using a CapsNet and 3D convolution, a three-dimensional capsule network (3D-CapsNet) model for recognizing the MI-EEG signal, and using an EEG signal in the 3D array form described in the S1 as an input of the 3D-CapsNet for recognizing the MI-EEG signal, wherein a 3D-CapsNet comprises a 3D convolution module and a CapsNet module; the 3D convolution module performs feature extraction on the input EEG signal in the 3D army form from both a temporal dimension and an inter-channel spatial dimension through a plurality of layers of 3D convolution to obtain a low-level feature; and the CapsNet module has a spatial detection capability, and the low-level feature output by the 3D convolution module is integrated through the CapsNet to obtain a high-level spatial vector containing an inter-feature relationship; and
S3: training the CapsNet module by using a dynamic routing algorithm, connecting a primary capsule and a motor capsule through dynamic routing, and finally outputting a classification result through a nonlinear activation function squash.

2. The method for recognizing an MI-EEG signal based on a CapsNet according to claim 1, wherein the step S1 comprises the following substeps:
intercepting the EEG signal by frame, obtaining a value of a current frame, transforming a value of each frame into an x×y 2D matrix (2D-map) based on a general spatial distribution of a sampled electrode, and filling an unused electrode position with 0; and
expanding TP 2D-maps into an x×y×TP 3D matrix based on temporal information of the EEG signal, wherein TP represents a quantity of sampling points for each channel, and TP is a natural number.

3. The method for recognizing an MI-EEG signal based on a CapsNet according to claim 2, wherein the step S2 is executed as follows: constituting the 3D convolution module by encapsulating five 3D convolution layers to extract a basic feature of the input EEG signal in the 3D array form at a plurality of levels to provide local perceptual information for a main capsule layer, and gradually increasing a quantity of convolution kernels to ensure that increasingly rich features are correctly extracted; performing batch normalization (BN) after each convolution to accelerate convergence and reduce overfitting; inputting the input into the convolution module to generate 128 4*5*6 outputs, converting the outputs into a 128*4*5*6 tensor, and sending the 128*4*5*6 tensor to the main capsule layer, such that the main capsule layer outputs 384 4-dimensional capsules, wherein the main capsule stores spatial features of different forms for the MI-EEG signal; connecting the main capsule layer and a motor capsule layer through the dynamic routing; aggregating, by the dynamic routing algorithm, predicted capsules that are similar to each other, and obtaining, through abstraction, a motor capsule capable of representing an inter-class difference; and outputting the classification result through the nonlinear activation function squash.

4. The method for recognizing an MI-EEG signal based on a CapsNet according to claim 3, wherein the step S3 is specifically as follows: training the CapsNet by using the dynamic routing algorithm, wherein an inter-capsule information transfer and routing process is only carried out between two consecutive capsule layers, that is, the dynamic routing algorithm is used between ûᵢⱼ and sⱼ; and a specific process is as follows:
$${\hat{u}}_{ij} = {u}_{i} {W}_{ij} \qquad ( 1 )$$
firstly, defining uᵢ(i=1, 2, . . . , n) to represent a detected low-level feature vector, and multiplying the low-level feature vector uᵢ by a corresponding weight matrix Wᵢⱼ to obtain a high-level output vector ûᵢⱼ, wherein i represents an iₜₕ low-level feature, and j represents a jₜₕ primary capsule; as shown in the formula (1), encoding a probability of a corresponding feature based on a vector length, and encoding an internal status of the feature based on a vector direction; and performing the above steps to encode a spatial relationship between the low-level feature and a high-level feature, wherein ûᵢⱼ is also referred to as the primary capsule;
secondly, weighting the primary capsule ûᵢⱼ, such that the capsule learns a coupled sparse weight cᵢⱼ by using the dynamic routing algorithm; adjusting the cᵢⱼ, and sending, by the primary capsule ûᵢⱼ, an output to an appropriate motor capsule sⱼ, wherein the sⱼ is a result of performing weighted summation on predicted vectors of a plurality of primary capsules, predicted values that are similar to each other are aggregated, and an entire process is shown in a formula (2):
$${s}_{j} = {\sum }_{i} {\hat{u}}_{ij} {c}_{ij} ; \qquad ( 2 )$$
and
finally, processing the sⱼ by using the nonlinear activation function squash, such that a length is compressed to within 0 to 1 without changing the vector direction, and a result is represented as a vector vⱼ, wherein as shown in a formula (3), the probability of the corresponding feature is encoded based on the vector length, and the internal status of the feature is encoded based on the vector direction:
$${v}_{j} = \frac{{‖ {s}_{j} ‖}^{2}}{1 + ‖ {s}_{j} ‖} \cdot \frac{{s}_{j}}{‖ {s}_{j} ‖} \qquad ( 3 )$$
wherein the above three steps are a complete inter-capsule propagation process, wherein learning of a coupling coefficient cᵢⱼ is an essence of the dynamic routing algorithm, and the coupling coefficient is determined according to the following formula (4):
$${c}_{ij} = \frac{exp ( {b}_{ij} )}{{\sum }_{k} exp ( {b}_{ik} )} \qquad ( 4 )$$
wherein bᵢⱼ represents a temporary variable with an initial value of 0; after a first iteration, all values of the coupling coefficient cᵢⱼ are equal; as the iteration progresses, a value of the bᵢⱼ is updated, and a uniform distribution of the cᵢⱼ changes; and the bᵢⱼ is updated according to the following formula (5):
$${b}_{ij} ← {b}_{ij} + {\hat{u}}_{ij} \cdot {v}_{j} . \qquad ( 5 )$$
